(19) United States
(12) Patent Application Publication
(10) Pub. No.: US 2005/0070803 A1
(43) Pub. Date: Mar. 31, 2005

(54) Multiphoton photoacoustic spectroscopy system and method

(76) Inventors: Brian M. Cullum, Laurel, MD (US); Nirmala Chandrasekharan, Baltimore, MD (US); Ronak Mehta, Reisterstown, MD (US); Soumi Saha, Edison, NJ (US)

Correspondence Address: FLESHNER & KIM, LLP, P.O. BOX 221200, CHANTILLY, VA, 20153, US

(21) Appl. No.: 10/721,824

(22) Filed: Nov. 26, 2003

Related U.S. Application Data
(60) Provisional application No. 60/507,353, filed on Sep. 30, 2003.

(51) Int. Cl. A61B 6/00

(52) U.S. Cl. 600/473; 600/476; 250/458.1; 356/303

(57) ABSTRACT

A system and method for performing multispectral imaging locates features of interest in a specimen using a technique known as multiphoton photoacoustic spectroscopy. In this technique, a tunable high-power laser is used to initiate multiphoton excitation events which are then detected as an acoustic signal using a sensor such as an ultrasonic piezoelectric transducer. The transducer signal is processed to form a normalized MPPAS signal intensity which may then be used as a basis for forming a spectral image. Unlike other spectroscopies, MPPAS is able to monitor non-fluorescent species based on non-radiative relaxation of the light-absorbing species in the specimen. In addition, since the majority of energy imparted to the light-absorbing molecules is released through non-radiative pathways, sensitive measurements of even fluorescent molecules can be performed. The system and method may be applied to detect malignant cells in tissue samples although other uses are contemplated.

### BACKGROUND OF THE INVENTION

1. Field of the Invention

This invention generally relates to medical imaging techniques, and more particularly to a system and method of performing chemical-based spectroscopy for non-invasively diagnosing anomalies or other features in specimens including but not limited to human tissue.

2. Description of the Related Art

In the last couple of decades, various non-invasive diagnostic techniques such as X-ray imaging, magnetic resonance imaging (MRI), ultrasound, positron emission tomography (PET), optical coherence tomography (OCT), elastic and diffuse reflectance, photoacoustics, fluorescence, Raman scattering, etc., have been employed to diagnose malignant tumors in vivo. Depending on the method employed to differentiate between normal and tumorous tissues, these different techniques can be classified as either morphological-based or chemical-based analyses.

Morphological-based methods such as X-ray, OCT, and ultrasound differentiate normal and tumorous tissues based on differences in densities between cancerous and non-cancerous tissues or on their water content. Because these techniques differentiate tissues based on tissue density, they are under certain conditions unable to accurately distinguish between dense healthy tissues and tumorous tissues.

Chemical-based techniques (i.e., fluorescence spectroscopy, etc.), on the other hand, differentiate normal and tumorous tissues by measuring differences in chemical composition (e.g., hemoglobin content and oxygenation level etc.). In order to perform such analyses, ultraviolet or blue light (300 nm to 450 nm) is typically required for excitation of the tissue, as these wavelengths have sufficient energy to excite the various chemical species being interrogated. Because of this limitation in excitation wavelengths and the strong broadband absorption properties of tissues in the ultraviolet and short wavelength visible region of the electromagnetic spectrum, such diagnoses are typically limited to layers of tissue less than 200 microns below the surface. These minimal penetration depths dramatically limit the applicability of chemical-based techniques for tumor diagnosis.

Recently, multiphoton fluorescence has been used in imaging formats to provide high-resolution chemical images of tissues for real-time tumor demarcation. In this technique, near infrared light in the spectral range known as the “diagnostic window” is focused down to a specific depth below the tissue surface. Within the focal volume, power densities are great enough to allow for multiphoton absorptions to occur, thereby exciting chemical constituents with ultraviolet or visible excitation spectra well below the surface of the tissue. Once excited, a small portion of these molecules relax to the ground state by the emission of fluorescent photons in the visible region of the electromagnetic spectrum. By monitoring differences in the fluorescence properties of normal and malignant tissues, either based upon different excitation or emission profiles, highly localized spatial analyses can be performed and three-dimensional images reconstructed of the chemical composition of the tissues.

The majority of fluorescence spectral differences between normal and malignant tissues are distinguished based on the short wavelength visible fluorescent photons that are generated. Unfortunately, photons at these wavelengths are often reabsorbed before reaching the tissue's surface. This absorption, in turn, limits the applicability of multiphoton fluorescence techniques to distances typically less than 200 microns.

### SUMMARY OF THE INVENTION

An object of the present invention is to provide a system and method of performing chemical-based spectroscopy which represents an improvement over other forms of imaging which have been proposed.

Another object of the present invention is to provide an improved system and method for non-invasively diagnosing anomalies or other features in specimens including but not limited to human tissue.

Another object of the present invention is to achieve one or more of the aforementioned objects through implementation of a non-invasive multiple-photon photoacoustic spectroscopy technique which diagnoses the presence of malignant tissue, biological molecules, and/or other anomalies and features at a subsurface level which extends to a depth of at least several millimeters under the tissue surface, and which also provides high-resolution chemical images of these features for analysis.

These and other objects and advantages of the present invention are achieved by providing a method for performing spectral imaging which includes generating multiple-photon excitation in a specimen, detecting photoacoustic waves resulting from the excitation, and forming a spectral image based on the photoacoustic waves. The multiple-photon excitation is generated based on simultaneous absorption of N photons by each of a plurality of species (e.g., molecules) in the specimen, where N≧2. This excitation is also preferably generated using solely unscattered (or so-called ballistic) photons directed towards the specimen, and from non-radiative relaxing that occurs from the light-absorbing species. The excitation generates photoacoustic waves either from non-fluorescent species in the specimen or both fluorescent and non-fluorescent species. These acoustic waves are detected, processed, and analyzed for locating anomalies or other features of interest that demonstrate an identifiable spectral signature. The specimen may be a tissue sample, a collection of biological molecules, or any other material capable of being spectrally imaged from multiple-photon induced excitation.

The present invention is also a system for performing spectral imaging which includes an exciter which generates multiple-photon excitation in a specimen and a detector which detects photoacoustic waves from the specimen as a result of the excitation. The exciter generates two-photon excitation in the specimen preferably based solely on unscattered photons. The photoacoustic waves generated by the excitation may derive from non-fluorescent species as well as fluorescent species. The detector maybe a piezoelectric transducer. In one illustrative implementation of this system, a test solution of aqueous rhodamine 6G was used to obtain a photoacoustic absorbency spectrum. The spectrum matched well with that of steady-state absorbency of the same solution. The system also showed a sensitivity to nanomolar concentrations of rhodamine 6G and below.

### BRIEF DESCRIPTION OF THE DRAWINGS

FIG. 1 is a diagram showing a system for performing multiphoton photoacoustic spectroscopy in accordance with one embodiment of the present invention.

FIG. 2 is a flow chart showing steps included in a method for performing multiphoton photoacoustic spectroscopy in accordance with an embodiment of the present invention.

FIG. 3 shows an energy level diagram of two-photon excitation induced in accordance with the present invention, which excitation includes a virtual state mediating the absorption process.

FIG. 4(a) is a diagram showing the manner in which 1 P excitation is generated, and FIGS. 4(b) and 4(c) show spectrum signals produced from the 1 P excitation caused by ballistic and scattered photons, which are diffuse in space and time.

FIG. 5(a) is a diagram showing the manner in which 2P excitation is generated in accordance with the present invention, and FIGS. 5(b) and 5(c) show spectrum signals produced from the 2P excitation caused by ballistic photons.

FIG. 6 is a diagram showing a system for performing multiphoton photoacoustic spectroscopy in accordance with another embodiment of the present invention.

FIG. 7(a) is a graph showing a wavelength-dependent spectrum for absorbency of a two micromolar concentration of rhodamine 6G produced by the system of FIG. 6, and FIG. 7(b) is a graph showing a UV-Visible absorbency spectrum for the same solution.

FIG. 8(a) is a graph showing a wavelength-dependent spectrum for absorbency of a two nanomolar concentration of rhodamine 6G produced by the system of FIG. 6, and FIG. 8(b) is a graph showing a UV-Visible absorbency spectrum for the same solution.

### DESCRIPTION OF THE PREFERRED EMBODIMENTS

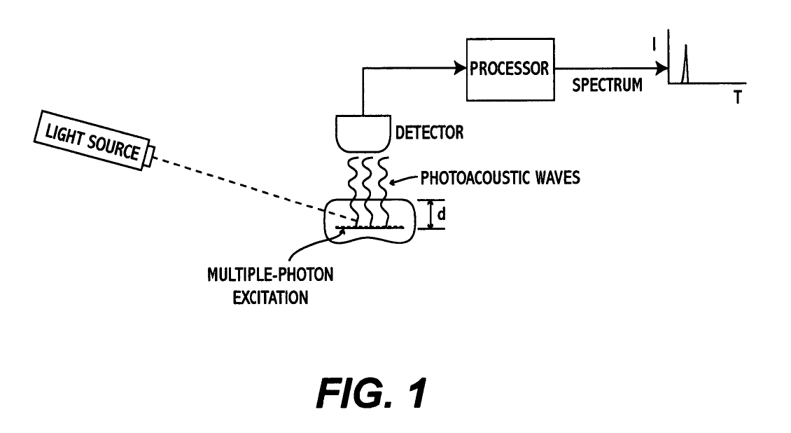

FIG. 1 shows a system for performing multiphoton photoacoustic spectroscopy (MPPAS) in accordance with one embodiment of the present invention. The system includes a light source 1, a detector 2, and a processor 3 which may be any type of general-purpose or specialized (e.g., ASIC or other chip-based) computing system. The light source is preferably a tunable high-power laser which generates a light beam within a predetermined range of wavelengths towards a specimen 5 to be analyzed. The wavelength range, laser power, or a combination of these or other parameters (e.g., objective lens power) may be selected to achieve a desired penetration depth for purposes of inducing multiple-photon excitation in the specimen accordance with the present invention, as described in greater detail below.

The detector maybe any type of transducer capable of sensing acoustic waves and converting them into electric signals. One example is a piezoelectric transducer (PZT) but those skilled in the art can appreciate that other sensors maybe used. The processor generates a spectral image from the signal output from the detector, after this signal is normalized to correct power fluctuations in the laser beam. The system may be used to analyze any type of material from which a spectrum may be generated based on photoacoustic wave emission. One advantageous but non-limiting application includes generating a spectral image of a tissue sample for purposes of detecting the presence of malignant cells. Other uses including performing spectral imaging for a collection of biological molecules.

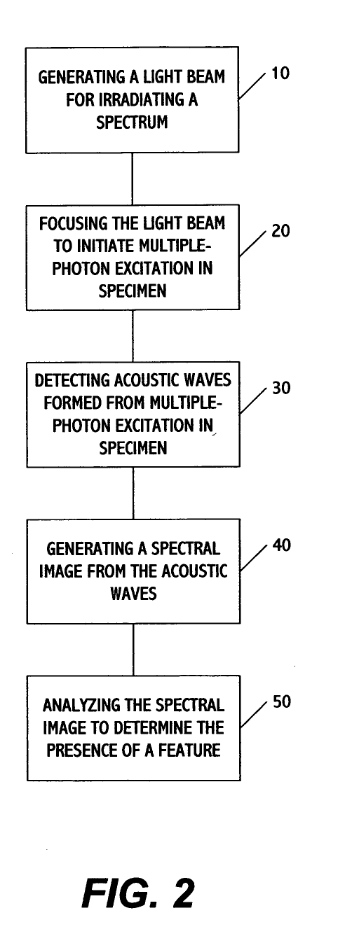

FIG. 2 is a flow chart showing steps included in a method for performing multiphoton photoacoustic spectroscopy using the system shown in FIG. 1. For illustrative purposes, it will be assumed that the specimen is a sample of human tissue suspected of possessing malignant cells. Based on this assumption, the method includes as an initial step generating a light beam for irradiating a specimen. (Block 10). The light beam is generated within a predetermined range of wavelengths that preferably corresponds to or includes wavelengths that lie within a diagnostic window of the tissue sample being tested.

The diagnostic window used very much depends on the analytes present in the tissue. This window may encompass, for example, the ultraviolet, visible, and/or near-infrared regions of the electromagnetic spectrum. Excitation wavelengths in the near infrared ≧800 nm would essentially cause multiphoton absorption to occur in the visible region ≧400 nm. Examples of analytes that absorb visible wavelengths are flavins, protoporphyrins, etc. If multiphoton excitation is in the visible wavelength range, then absorption occurs is in the ultraviolet region of the electromagnetic spectrum. Examples of the analytes in tissues that absorb in the ultraviolet are nicotinamide adenine dinuleotide, tryptophan, tyrosine, etc. Laser powers that may be used for the aforementioned wavelengths range from microwatts to megawatts, depending on the temporal pulse width of the laser as well as the desired penetration depth and type of tissue investigated.

A second step includes focusing the light beam to induce multiple-photon excitation at a predetermined depth in the tissue sample. (Block 20). Focusing maybe accomplished using any one of a variety of optical components. In one exemplary embodiment discussed in greater detail below, a high-power objective lens is used to induce two-photon excitation in the tissue sample. Depending on the sample under test and resolution desired, three-photon excitation or more may alternatively be initiated. Through selection of the wavelength range and/or the laser power, the multiple-photon radiation may advantageously be induced at a depth ranging from micrometers to approximately a centimeter.

More specifically, the penetration depth is dependent on the wavelength of excitation as well as on the type of sample under test. In preliminary studies, in which aqueous rhodamine dye was exicted using near-infrared wavelengths discussed in greater detail below, a penetration depth of >0.5 cm was easily achieved. For shorter wavelengths, the penetration depth would be lower since the scattering is inversely proportional to the fourth power of the wavelength of excitation light employed. Thus the higher the scattering, the lower the penetration depth. In the case of tissues, employing near-infrared light may achieve a penetration depth ranging from micrometers to approximately one centimeter, depending on the scattering coefficient of the particular tissue. At these depths, there is sufficient penetration to detect cancer cells that cannot be detected by other types of spectral imaging methods.

A third step includes detecting photoacoustic waves formed as a result of the multiple-photon excitation generated in the specimen. (Block 30). The photoacoustic waves form when excited species in the specimen (including those located at a position coincident with the maximum penetration depth of the beam) return to steady state. When this occurs, acoustic waves are generated which pass through the surface of the specimen where they are detected and converted into electrical signals by the detector.

A fourth step includes processing the detector signal to generate a spectral image. (Block 40). This step preferably includes processing the signal using known techniques to correct fluctuations in power of the laser beam, thereby resulting in the generation of a normalized spectral signal which can be viewed on an oscilloscope or other imaging device.

A fifth step includes analyzing the spectrum of the normalized signal to determine the presence of malignant cells or other types of anomalies. (Block 50). The spectrum analysis maybe performed using any one of a variety of techniques. One technique is based on chemical differentiation between cancerous and non-cancerous tissues. First, the absorption spectra of the different analytes in a tissue (e.g., flavins, tryptophan, tyrosine etc.) are recorded. Differences in the relative intensities of the analytes in cancerous and non-cancerous tumors, as well as differences in the shapes of their absorption profiles, are then identified and these will serve as markers for differentiating between cancerous and non cancerous tissues. Other techniques involve performing performing a peak analysis at predetermined wavelengths in the spectrum.

As mentioned, MPPAS is performed by inducing multiphoton excitation/absorption events in a specimen followed by an acoustic/ultrasonic detection event. When describing the signals generated by this MPPAS technique and the factors influencing these signals, the excitation and detection phases maybe treated independently for most cases.

### Two-Photon (2P) Absorption

For 2P excitation to occur, two photons must be simultaneously absorbed by each of a plurality of species (e.g., molecules) in the specimen. Therefore, the number of excited molecules is proportional to the square of the intensity. In addition, the excitation wavelength is doubled relative to single-photon absorption (i.e., λ_{exc(2P)}=2λ_{exc(1P)}) and the molecular 2P-absorption rate constant is given by k₂ₚ=σ₂ₚ (I/hΩ)², where I/(hΩ) is the number of photons/second×cm² and σ₂ₚ is the 2P-absorption cross-section.

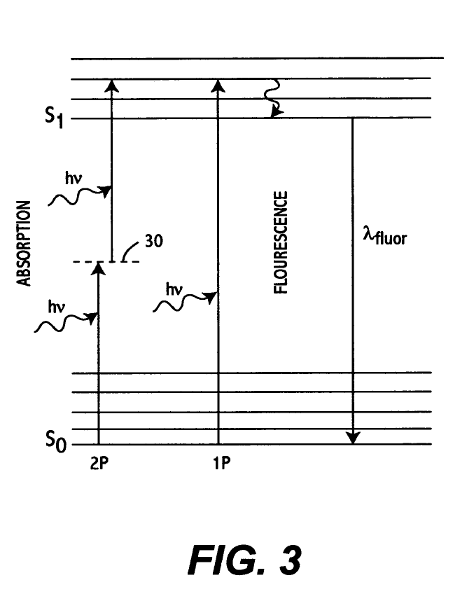

FIG. 3 is an energy level diagram showing one way 2-photon transition may take place in accordance with the present invention. In this diagram, the energy of a specimen molecule is initially shown as residing at steady state S₀. When irradiated with the laser beam, the molecule simultaneously absorbs the energy of two photons. During this absorption process, the energy each photon imparts to the molecule (represented as hν) excites the molecule up to an elevated energy level represented by S₁. The excitation energy in the molecule is dissipated as heat through molecular collisions with surrounding molecules. These collisions, in turn, create a pressure wave (e.g., an acoustic or ultrasonic wave) that can then be measured with a piezoelectric device or some other type of acoustic or ultrasonic transducer. After all the energy is dissipated, the molecule returns to the steady state.

Under conditions of weak light scattering, for optically thin samples and when all the absorbed 2P excitation is rapidly converted to heat, the MPPAS signal intensity, V_{pzt}, is proportional to cI{1−[exp(−αl)]} where I is the incident laser pulse energy, c is an instrumental constant, α is the absorptivity of the sample at the wavelength of interest, and I is the effective path length through the sample at the laser wavelength. For the two-photon process, αl is small and hence this relationship can be simplified to V_{pzt}∝cIαl. Therefore, the amplitude of the observed photoacoustic waveform is directly proportional to the absorptivity of the sample at any given laser wavelength.

The 2P absorption performed in accordance with the present invention is compared with one-photon (1P) absorption in FIG. 3. As shown, during 1 P absorption a single photon (hν) excites a molecule from steady state to the excited state. When this photon returns to level S₀, visible fluorescent light (λ_{fluor}) is emitted which can be detected for generating a spectral image. However, in the 2P process of the present invention a first photon elevates the molecule to a viral mediating state illustratively shown by line 30, and a second photon performs the remaining excitation up to the S₂ level. This two-step elevation process occurs as a function of photon density.

More specifically, when tightly focused, pulsed lasers produce such high photon densities that simultaneous absorption of two photons occurs. This multiple absorption event (unlike in 1P where only one photon is absorbed) induces a molecular excitation of a magnitude equivalent to the absorbed photon energies, with each photon in the 2P process having half the energy of the single photon in the 1P process. This absorption/excitation process produces the collisions previously described, which ultimately results in the formation of acoustic waves which are detected by the present invention for purposes of generating a spectral image.

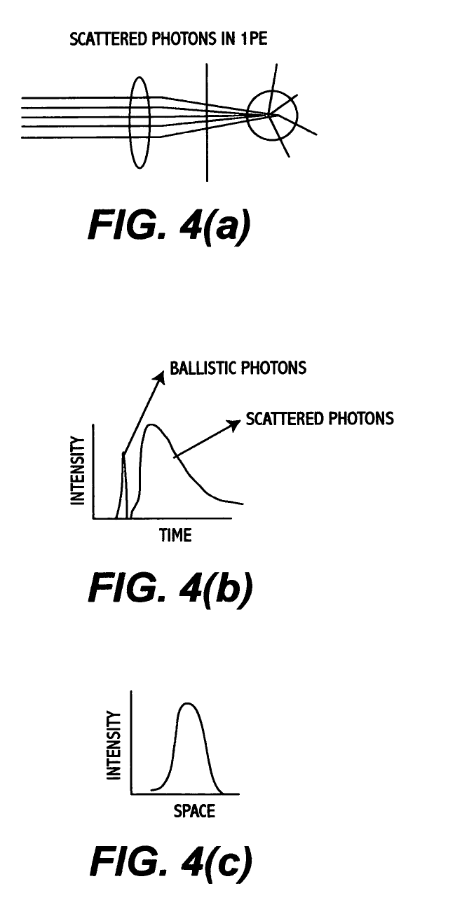

When exciting a specimen through the 2P process, several key differences are observed relative to single-photon excitation events. One fundamental difference between 2P and 1P excitation is based on the type of photons used to induce excitation. In the 1 P process, both scattered and non-scattered photons are used to induce excitation. See FIG. 4(a), where the scattered photons are shown by reference numeral 35 and FIG. 4(b) are spectral diagrams showing that the scattered photons are diffuse in space and time.

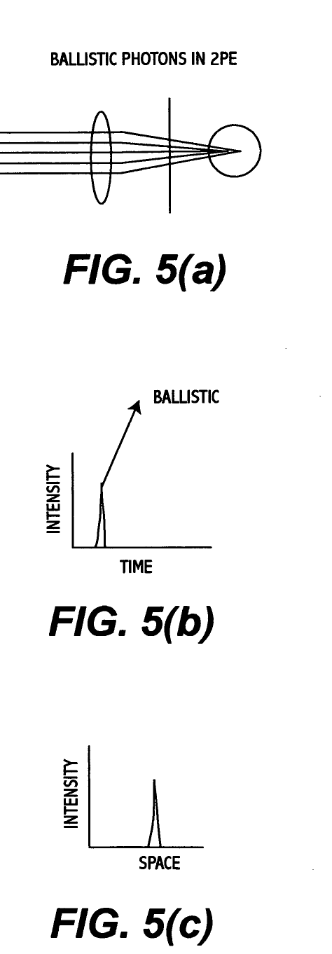

In the 2P process, only non-scattered or ballistic photons contribute significantly to the excitation process. (See FIG. 5(a) where the scattered photons in the 1P process do not exist.) These ballistic photons are present only in the direct path of the laser beam and therefore are sensitive to the position of the laser beam. Hence, 2P absorbency occurs exclusively at the focal point of the laser. As a result, 2P excitation typically results in a highly localized absorption within scattering samples. (This advantage is clearly evident in FIG. 5(b), where in comparison with the 1P process a sharp and distinctly defined spectrum signal produced from ballistic-photon excitation is shown.) This localized excitation provides the potential for obtaining greater penetration depths as well as highly resolved chemical images of samples.

### Photoacoustic Wave Detection

The photoacoustic spectroscopy performed by the present inventions a sensitive technique that provides information about the spectral absorption profile of a chemical species. As indicated, during this process a pulsed (or modulated) tunable excitation source is used to excite a particular chemical species. When that species absorbs the wavelength of light used for excitation (which, in this case is 2P excitation), the energy deposited within the molecules is dissipated as heat through molecular collisions with surrounding molecules. These collisions, in turn, create a pressure wave (e.g., an acoustic or ultrasonic wave) that can then be measured with a piezoelectric device or some other type of acoustic or ultrasonic transducer. By scanning the excitation source over a series of wavelengths and monitoring the magnitude of the transducer signal, the present invention is able to measure the absorption spectrum of the 2P-excited sample.

The present invention thus combines the strengths of acoustic detection (e.g., low background, good signal transmission, and deep tissue penetration) with those of two-photon excitation (i.e., deep tissue penetration and a high degree of spatial localization) for spectral monitoring of various chemical species. Using excitation wavelengths within the diagnostic window of tissues and a two-photon absorption scheme, it is possible to achieve excitation of absorbing species up to several millimeters below the surface of the tissue.

More specifically, unlike other spectral imaging techniques such as fluorescence spectroscopy (where a visible photon is produced and must travel back to the surface without being absorbed), MPPAS relies on acoustic (ultrasonic) waves reaching the surface. Because of the minimal attenuation of ultrasonic waves in tissue, over depths of ≦1 cm, the present invention is able to monitor chemical species much deeper in the tissue than fluorescence techniques and with much greater spatial resolution and chemical information. MPPAS is therefore ideally suited for subsurface tumor diagnosis as well as tumor margining during surgical removal.

MPPAS is advantageous for a number of other reasons. For example, because a large portion of most excited molecules relax through a thermal decay process, photoacoustic detection is an extremely sensitive means of detection compared with other spectral imaging techniques. In addition, because the excitation source is light instead of pressure, minimal background signal is present, which in turn allows for a lower detection limit to be achieved.

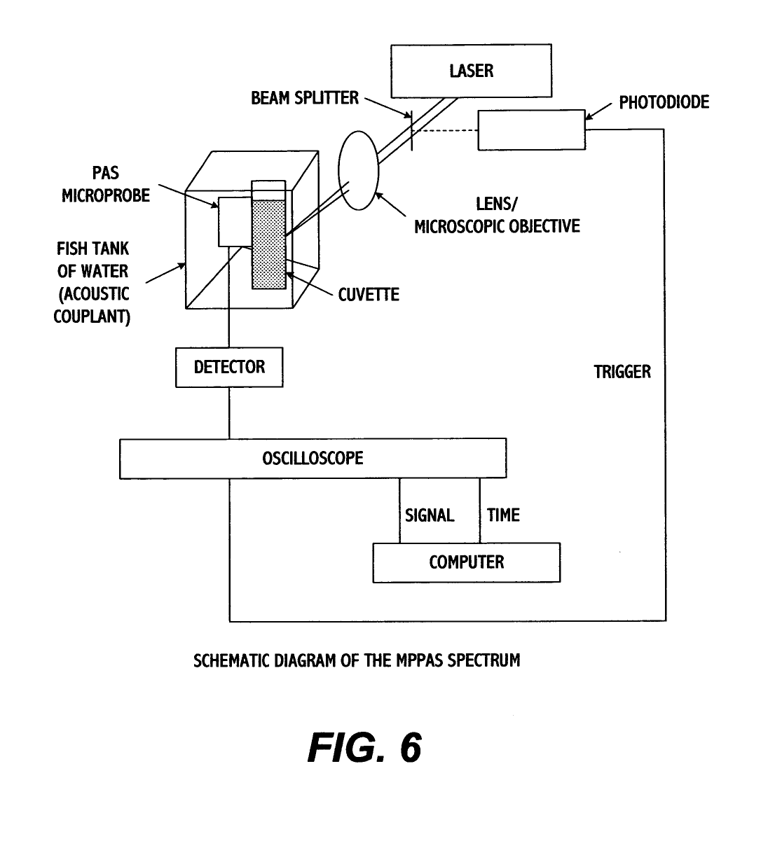

FIG. 6 shows an exemplary embodiment of a system that was used to test performance of the multiphoton photoacoustic spectroscopic technique in accordance with the present invention. The system includes an excitation source 50, a detector 60, oscilloscope 70, and a computer 80 which were applied to test a standard dye, rhodamine 6G (R6G) sample held within a cuvette 90. R6G was chosen as a test sample because of its well-characterized absorption profile as well as its ready accessibility, thus making it convenient and easy to compare the MPPAS spectrum for R6G with that of the UV-V is absorbency spectrum obtained for the same sample.

The excitation source was a tunable, pulsed Nd:YAG pumped optical parametric oscillator (OPO; Opotek Inc., Vibrant B) with output powers of ≧20 mJ per pulse. The wavelength range employed was in the near-IR, between 980-1100 nm. In order to achieve a 2P absorption event, the laser beam was focused onto the sample using a 10×microscope objective/lens 52. This produced excitation at a depth of more than 0.5 cm for the aqueous and gelatin embedded rhodamine 6G sample using the near-infrared light. In accordance with the present invention, the penetration depth maybe varied as desired by adjusting one or more of the wavelength of the laser, density, scattering coefficient, vascularization, etc. of the tissue. Following sample absorption, the resulting photoacoustic wave was coupled to the detector (e.g., a commercial piezoelectric transducer) using water as an acoustic couplant held within a tank 100.

The photoacoustic signal was amplified using an impedance-matched pre-amplifier before being recorded and averaged on the oscilloscope (which was a 500 MHz digital sampling oscilloscope) for a predetermined number of sweep cycles. Simultaneous to the recording of ultrasonic signals, laser powers were also measured using a photodiode 120. The output of this photodiode was then recorded in another channel of the same digital oscilloscope. The photodiode signals were then used to correct the measured MPPAS signals for laser power fluctuations associated with shot-to-shot variations in the laser power, as well as any wavelength dependent laser power variations.

Specific non-limiting values used to implement this exemplary embodiment include a 2×10⁻⁶ M aqueous solution of R6G, which was prepared and placed in the glass cuvette. The microscope objective, used for 2P excitation of the sample, focused the laser beam to the center of the cuvette. The resulting photoacoustic signal generated from the rhodamine absorption traveled a distance of 0.5 cm (half the path length for a 1 cm cuvette) before reaching the location of the ultrasonic transducer. Since the absorbency by R6G is instantaneous after the onset of the laser pulse, the delay time of the subsequent photoacoustic signal generated was calculated to be ≧14.2 ms after laser excitation. The time response of the MPPAS signal (trace) was collected for multiple wavelengths, between 980-1100 nm and these wavelength dependent time responses were used to construct MPPAS spectra.

To obtain the total MPPAS signal generated by R6G, the computer integrated the sinusoidal photoacoustic wave generated for a time interval of 25 μs (5 μs after laser excitation up to 30 μs). The integrated intensity of the MPPAS signal was then calculated at each wavelength and further normalized for laser power fluctuations. This normalization was performed, first, by plotting the wavelength dependent spectrum of the laser intensity from the photodiode data. This was followed by choosing the wavelength which yielded the highest laser intensity, and ratioing this intensity to the intensities at all other wavelengths in the spectral range of interest. The MPPAS signal recorded at each wavelength was then divided by this value.

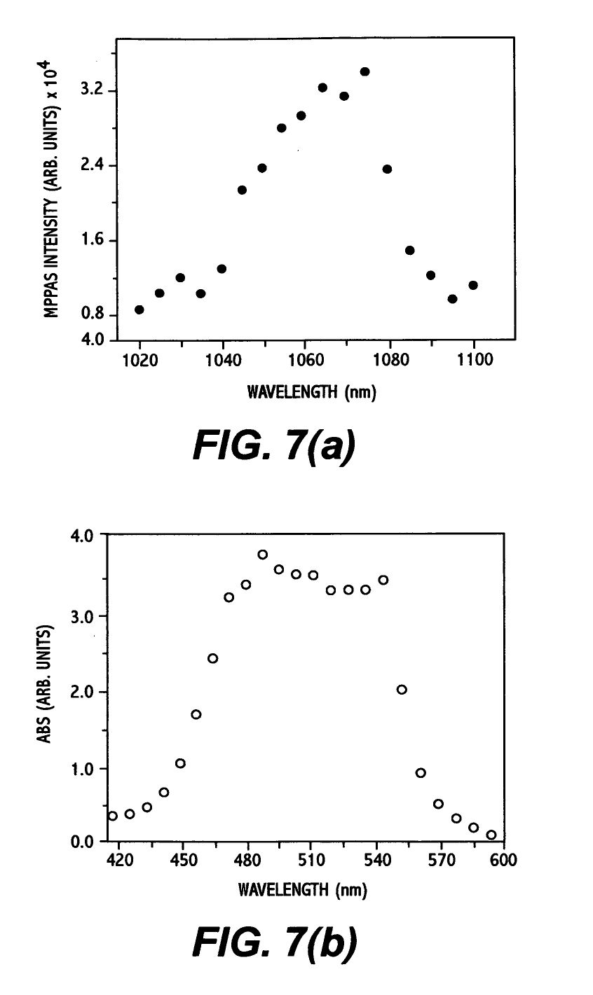

FIG. 7(a) shows the MPPAS spectrum obtained for a micromolar concentration solution of an R6G sample (solid circles). The absorbency maximum occurs at 1070 nm (535 nm in single photon absorbency) originating from the R6G monomers. The absorbency of R6G (open circles) recorded with a commercial UV-Visible spectrometer is shown in FIG. 7(b) for comparison. It may be seen that the absorbency obtained from the photoacoustic signal is in good agreement with that of the steady state absorbency. R6G has a fluorescence quantum yield of approximately 0.5 in water. Therefore, approximately 50% of the molecules relax radiatively and approximately 50% relax non-radiatively. However, even at 50% efficiency, the photoacoustic signal of our micromolar R6G sample is strong and easy to measure. For most tissue chromophores, the fluorescence quantum yields are approximately 0.1-0.2. This implies that nearly 80-90% of the molecules are available for non-radiative relaxation. Hence, it is expected that the photoacoustic signal (which originates via non-radiative relaxation) would be even more dramatically enhanced for tissues and biological molecules.

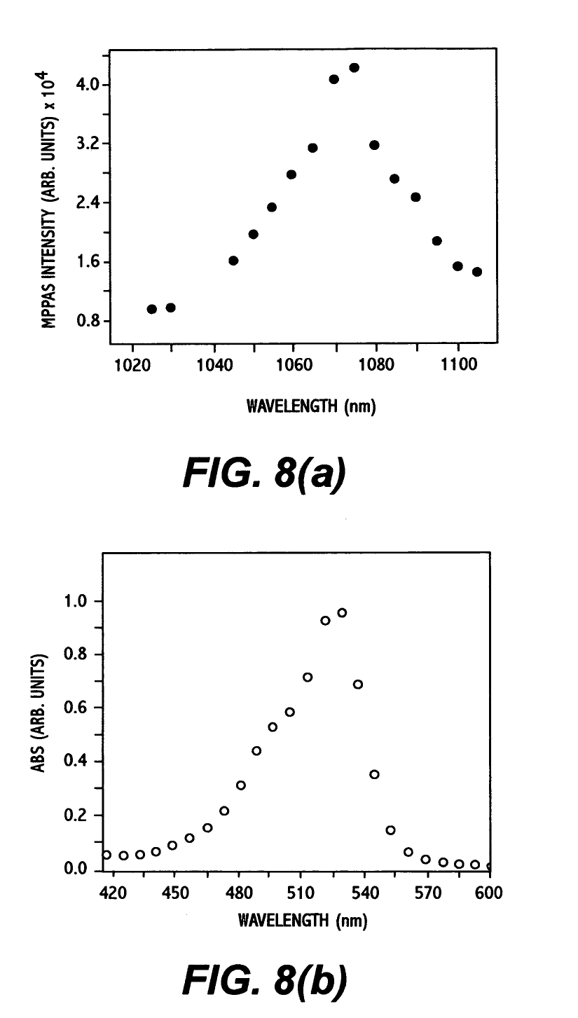

In order to determine the sensitivity of the technique of the present invention, MPPAS was performed on a very dilute nanomolar solution of R6G. As shown by the solid circles in FIG. 8(a), the spectrum produced by this sample indicates that an absorbency maximum occurs at approximately 1075 nm. By comparison, the absorbency maximum for a corresponding single-photon-excited spectrum (shown in FIG. 8(b) by open circles) occurs at approximately 530 nm.

Some important differences may be seen between the spectra in FIGS. 7 and 8. For instance, the MPPAS spectrum for the micromolar concentration of R6G (FIG. 7(a)) is slightly lower in intensity when compared to that of the MPPAS spectrum of the nanomolar concentration of R6G (FIG. 8(a)). The reverse is true in the case of the steady state absorbency spectra of FIGS. 7(b) and 8(b). While it is well known that steady-state absorbency increases with increasing concentration of the sample since the total number of absorbing molecules increase this is not always the case for MPPAS. To illustrate, in this case, the intensity of the MPPAS spectrum decreases with increased concentration of R6G. This can be explained by reduction in ballistic photons at the higher concentration solution, thus decreasing the potential for a 2P excitation. Therefore, at lower concentrations, the sensitivity of MPPAS can actually increase in some cases.

The present invention has therefore provided for the first time a non-invasive spectroscopic technique known as MPPAS. This technique has been developed and characterized using a test sample of rhodamine 6G. Using this technique, spectra have been obtained for two different concentrations of R6G. They show agreement with steady state absorbency spectra obtained from a UV-V is spectrometer. In addition, this technique has proved to be sensitive (e.g., capable of monitoring nanomolar R6G). As R6G has a fluorescence quantum yield of approximately 0.5 in water, it is expected that the detection of less fluorescent species should be dramatically enhanced through the present invention. MPPAS therefore serves as a tremendously effective tool for the subsurface imaging of chemical/molecular differences in tissues.

Other modifications and variations to the invention will be apparent to those skilled in the art from the foregoing disclosure. Thus, while only certain embodiments of the invention have been specifically described herein, it will be apparent that numerous modifications maybe made thereto without departing from the spirit and scope of the invention.

1. A method for performing spectral imaging, comprising:
generating multiple-photon excitation in a specimen;
detecting photoacoustic waves resulting from the excitation; and
forming a spectral image based on the photoacoustic waves.

2. The method of claim 1, wherein the multiple-photon excitation is generated based on simultaneous absorption of N photons by each of a plurality of species in the specimen, where N≧2.

3. The method of claim 2, wherein the generating step includes:
directing unscattered photons on the specimen to generate the multiple-photon excitation.

4. The method of claim 3, wherein the multiple-photon excitation is generated solely as a result of directing the unscattered photons onto the specimen.

5. The method of claim 1, wherein the photoacoustic waves derive from non-radiative relaxing light-absorbing species in the specimen.

6. The method of claim 1, wherein the photoacoustic waves derive from non-fluorescent species in the specimen.

7. The method of claim 1, wherein the photoacoustic waves derive from fluorescent and non-fluorescent species in the specimen.

8. The method of claim 1, wherein the generating step includes:
irradiating the specimen with light to a predetermined depth and within a predetermined range of wavelengths.

9. The method of claim 8, wherein the specimen is tissue.

10. The method of claim 9, wherein the predetermined depth is several millimeters.

11. The method of claim 10, wherein the predetermined wavelength range includes wavelengths lying within a diagnostic window of the tissue.

12. The method of claim 1, wherein the specimen is tissue.

13. The method of claim 1, wherein the specimen is a collection of biological molecules.

14. The method of claim 1, wherein the photoacoustic waves include ultrasonic waves.

15. The method of claim 1, further comprising:
analyzing the spectral image to detect a feature within the specimen.

16. The method of claim 15, wherein the feature is malignant tissue.

17. The method of claim 1, wherein the multiple-photon excitation is two-photon excitation in the specimen.

18. A system for performing spectral imaging, comprising:
an exciter which generates multiple-photon excitation in a specimen; and
a detector which detects photoacoustic waves from the specimen as a result of the excitation.

19. The system of claim 18, wherein the exciter generates two-photon excitation in the specimen.

20. The system of claim 19, wherein the exciter generates two-photon excitation in the specimen based solely on unscattered photons.

21. The system of claim 18, wherein the exciter includes:
a laser which directs light within a predetermined range of wavelengths into the specimen.

22. The system of claim 21, wherein said predetermined range of wavelengths causes the light to penetrate a predetermined depth into the specimen.

23. The system of claim 22, wherein the specimen is tissue.

24. The system of claim 23, wherein said predetermined depth is several millimeters.

25. The system of claim 22, wherein said predetermined range of wavelengths includes wavelengths lying within a diagnostic window of the tissue.
